(19) United States
(12) Patent Application Publication
(10) Pub. No.: US 2021/0030317 A1
(43) Pub. Date: Feb. 4, 2021

(54) ENZYME-FREE GLUCOSE DETECTION CHIP FOR DETECTING BLOOD SUGAR LEVEL OF A BLOOD IN A NEUTRAL ENVIRONMENT

(71) Applicant: National Chung Hsing University, Taichung City (TW)

(72) Inventors: GOU-JEN WANG, Taichung City (TW); PO-YU PENG, Taichung City (TW); FANG-YU LIN, Taichung City (TW)

(21) Appl. No.: 17/033,388

(22) Filed: Sep. 25, 2020

Related U.S. Application Data
(63) Continuation-in-part of application No. 15/414,599, filed on Jan. 24, 2017.

(30) Foreign Application Priority Data
Jan. 29, 2016 (TW) 105102964

(51) Int. Cl. A61B 5/1473; A61B 5/145; G01N 27/327

(52) U.S. Cl. CPC A61B 5/1473; G01N 27/3278; A61B 5/14532

(57) ABSTRACT

Disclosed in the present invention is an enzyme-free glucose detection chip for detecting blood sugar level of a blood in a neutral environment, including: a substrate; a detection portion, disposed on an end surface of the substrate; a plurality of protrusions, disposed at the detection portion; a conductive layer, disposed on a surface of the substrate having the protrusions; and a plurality of gold nanoparticles, dispersed on surfaces of the protrusions, wherein the characterized in that the conductive layer is made by mixing gold and platinum and the ratio (mol/v) of platinum to gold is 1:2 to 4:1.

### BACKGROUND OF THE INVENTION

### 1. Field of the Invention

The present invention relates to a detection tool, and more particularly to an enzyme-free glucose detection chip for detecting blood sugar level of a blood in a neutral environment.

### 2. Description of the Related Art

Nowadays, diabetes is the most severe health problem all over the world. The International Diabetes Federation points out that, up to 2015, globally approximately 3.87 billion people are facing the threat of diabetes, of which 90% are patients with type II diabetes. Type II diabetes means that the body cannot fully use insulin produced by the pancreas, resulting in unstable blood sugar in the body. Therefore, for a patient with type II diabetes, it is very important to detect blood sugar regularly to maintain a blood sugar level.

Currently, many technologies are used to perform continuous monitoring of blood sugar. Generally, an electrochemical method and an optical method are commonly used technologies. Based on advantages such as a low cost, high applicability, and simple operations of an electrochemical sensor, the electrochemical sensor is currently the most commercially acceptable tool. Furthermore, for an existing electrochemical sensor, an enzymatic method is used to achieve an objective of detecting glucose concentration. In the enzymatic method, through catalysis of glucose-specific glucose oxidase, glucose is oxidized into gluconolactone. Advantages of the enzymatic method are high reactivity toward glucose and high specificity for glucose detection. However, the use of glucose oxidase has the following disadvantages: A large number of complex fixed steps are required, thermal stability and chemical stability are undesirable, and degradability is high.

Based on the disadvantages of the enzymatic method, currently, consumables such as a blood sugar test strip required to detect blood sugar have high manufacturing costs and are difficult to store. If glucose oxidase degrades because of an environmental factor, the detection result may be erroneous. In other words, the current blood sugar detection technology developed on the basis of the enzymatic method still has considerable room for improvement.

### SUMMARY OF THE INVENTION

A major objective of the present invention is to provide an enzyme-free glucose detection chip, which can detect the blood sugar level from a blood directly in a neutral environment, so that it does not need to pretreat the blood before reacting with the enzyme-free glucose detection chip. Therefore, detecting the blood glucose level by the enzyme-free chip can save more time and money.

Another objective of the present invention is to provide the enzyme-free glucose detection chip, which can reacting with the glucose for generating the energy by itself, so that the enzyme-free glucose detection chip can be working without provided an exterior power.

Moreover, it can detect the blood glucose directly in the neutral environment, the enzyme-free glucose detection chip of this invention does not only be used for detecting a blood sample, but also can be implanted into a subject's body for continuously reacting and detecting the blood sugar level of the subject.

Another objective of the present invention is to provide the enzyme-free glucose detection chip, which can directly and accurately detect glucose concentration in the neutral environment without glucose oxidase, so that an error caused by deterioration of an enzyme of a test strip in conventional blood sugar detection can be reduced.

Another objective of the present invention is to provide a method for massively preparing enzyme-free glucose detection chips, which can achieve the efficacy of stable quality, a simplified manufacturing procedure, and a reduced cost.

To achieve the foregoing objective, disclosed in the present invention is an enzyme-free glucose detection chip for detecting blood sugar level of a blood directly in a neutral environment, the enzyme-free glucose detection chip.

Furthermore, the enzyme-free glucose detection chip includes: a substrate; a detection portion, disposed on an end surface of the substrate; a plurality of protrusions, disposed at the detection portion; a conductive layer, disposed on a surface of the substrate having the protrusions; and a plurality of gold nanoparticles, dispersed on surfaces of the protrusions; wherein the conductive layer is made by mixing gold and platinum in a predeterminate ratio.

In the embodiments of the invention, the conductive layer is made by mixing gold and platinum in predeterminate ratio (mol/v) of platinum to gold is 1:2 to 4:1, for example, the predeterminate ratio (mol/v) of platinum to gold may be 1:1 2:1, 3:1 or 4:1.

In one embodiment of the invention, the blood is from a subject, such as a tube of blood sample. The PH value of the blood is neutral, about 7.3-7.4, and the blood can directly react with the enzyme-free glucose detection chip without adjusting PH value of the blood.

In one embodiment of the invention, the enzyme-free glucose detection chip is implanted into a subject's body for detecting the blood sugar level of the blood.

In another embodiment of the invention, the enzyme-free glucose detection chip further including a power generation portion made by alloy of gold and platinum.

Preferably, each protrusion is semispherical.

Preferably, each protrusion is columnar.

Preferably, each protrusion has a micrometer-level size.

Preferably, each protrusion has a diameter between 1 micrometer and 20 micrometers. For example, each protrusion has a diameter of 1 micrometer, 2 micrometers, 5 micrometers, 10 micrometers, 12 micrometers, 15 micrometers or 20 micrometers.

Preferably, each gold nanoparticle has a diameter between 2 nanometers and 100 nanometers. For example, each gold nanoparticle has a diameter of 2 nanometers, 4 nanometers, 6 nanometers, 8 nanometers, 10 nanometers, 20 nanometers, 30 nanometers, 40 nanometers, 50 nanometers, 60 nanometers, 70 nanometers, 80 nanometers, 90 nanometers or 100 nanometers.

Moreover, disclosed in the present invention is a method for massively preparing the foregoing enzyme-free glucose detection chips, wherein the method includes the following steps:

step a: taking a base material, and coating a surface of the base material with a photoresist coating;

step b: treating the base material by using a photolithography technology, so that the base material includes a plurality of detection portions, and each detection portion has a photoresist array;

step c: sputtering a film made by mixing gold and platinum on a surface of the base material having the photoresist array, wherein the ratio (mol/v) of platinum to gold is 1:2 to 4:1;

step d: cutting the base material into a plurality of substrates, where each substrate includes a detection portion;

step e: evenly dispersing gold nanoparticles on surfaces of the photoresist arrays; and

step f: obtaining a massive quantity of enzyme-free glucose detection chips.

Preferably, the method further includes a thermal melting step between step b and step c: deforming the photoresist array with a temperature higher than a glass-transition temperature of a photoresist.

Preferably, the method further includes a packaging step between step e and step f: covering a region other than the detection portion on the substrate with a packaging layer.

Preferably, the method further includes a packaging step between step e and step f: covering a region other than the detection portion on the substrate with a packaging layer.

### BRIEF DESCRIPTION OF THE DRAWINGS

FIG. 1A is a schematic diagram of cleaning a silicon chip and a photoresist coating.

FIG. 1B is a schematic diagram of exposure and development.

FIG. 1C is a schematic diagram of a thermal melting step.

FIG. 1D shows sputtering of a gold film and disposal of gold nanoparticles.

FIG. 2A is a schematic diagram of etching a plurality of detection portions on a large substrate.

FIG. 2B is a schematic diagram of packaging of an enzyme-free glucose detection chip disclosed in the present invention.

FIG. 2C shows that each protrusion disposed in the present invention is a micrometer/nanometer composite structure.

FIG. 3A shows a semispherical array after a gold sputtering step observed from a viewing angle of 45 degrees.

FIG. 3B is an enlarged view of a height of a single protrusion.

FIG. 3C shows a result of observing a protrusion array after the step of disposing gold nanoparticles.

FIG. 3D shows a result of observing a single protrusion after the step of disposing gold nanoparticles.

FIG. 4A shows cyclic voltammograms of protrusions disclosed in the present invention and a normal gold electrode, where a line A represents the normal gold electrode, and a line B represents the enzyme-free glucose detection chip disclosed in the present invention.

FIG. 4B shows current-time curves of the protrusions disclosed in the present invention and the normal gold electrode according to FIG. 4A, where a line A represents the normal gold electrode, and a line B represents the enzyme-free glucose detection chip disclosed in the present invention.

FIG. 4C shows a cyclic voltammogram obtained by analyzing, through cyclic voltammetry at a different scan rate, the enzyme-free glucose detection chip disclosed in the present invention.

FIG. 4D shows a linear relationship between a square root of a scan rate and a current, where a lower line represents the normal gold electrode, and a upper line represents the enzyme-free glucose detection chip disclosed in the present invention.

FIG. 5A shows a cyclic voltammogram obtained by performing detection, through cyclic voltammetry at different glucose concentration, on the enzyme-free glucose detection chip disclosed in the present invention.

FIG. 5B shows a result obtained by performing a current analysis method on the enzyme-free glucose detection chip disclosed in the present invention, where a related standard curve is shown in a square box.

FIG. 6 shows a detection result obtained when glucose, ascorbic acid, and glucose are sequentially injected onto the enzyme-free glucose detection chip disclosed in the present invention.

FIG. 7 shows a result of detecting stability of the enzyme-free glucose detection chip disclosed in the present invention.

FIG. 8 is a schematic diagram of the the enzyme-free glucose detection chip in another embodiment of this invention.

FIG. 9A shows the results for detecting glucose by the enzyme-free glucose detection chips with the conductive layer made by mixing platinum and gold in different ratio (mol/v).

FIG. 9B shows a bar graph of the results for detecting glucose by the enzyme-free glucose detection chips with the conductive layer made by mixing platinum and gold in different ratio (mol/v).

FIG. 9C shows a result of detecting stability of the enzyme-free glucose detection chips with the conductive layer made by mixing platinum and gold in different ratio (mol/v).

FIG. 9D shows a result of detecting sensitivity of the enzyme-free glucose detection chips with the conductive layer made by mixing platinum and gold in different ratio (mol/v).

### DETAILED DESCRIPTION OF THE PREFERRED EMBODIMENTS

Disclosed in the present invention an enzyme-free glucose detection chip, including: a substrate; a detection portion, disposed on an end surface of the substrate; a plurality of protrusions, evenly distributed at the detection portion; a conductive layer, disposed on a surface of the substrate having the protrusions; and a plurality of gold nanoparticles, evenly distributed on surfaces of the protrusions; wherein the characterized in that the conductive layer is made by mixing gold and platinum and the ratio (mol/v) of platinum to gold is 1:2 to 4:1. Therefore, the enzyme-free glucose detection chip can detect the blood sugar level of a blood directly in a neutral environment.

The blood of this invention may be a blood sample isolated from an animal, such as human, or mean a blood tissue of a subject. That means the enzyme-free glucose detection chip can react and detect the blood sugar value of the blood in vivo or in vitro.

In the enzyme-free glucose detection chip disclosed in the present invention, the conductive layer coating on the surface of each protrusion to make the protrusions be used as electrodes for directly detecting the current or/and potential value when the PH value of the blood is neutral to react with the enzyme-free glucose detection chip. The gold nanoparticles can directly react with glucose without any glucose oxidase or/and any medium.

The enzyme-free glucose detection chip disclosed in the present invention is prepared through photolithography, a photoresist thermal melting method, and a step of sputtering a gold film.

First, through photolithography, a photoresist array is provided on the substrate. Subsequently, the photoresist thermal melting method is performed, where the photoresist array is heated and softened. Next, the step of sputtering a gold film is performed to provide a gold nano-film on the substrate. Subsequently, gold nanoparticles are disposed on surfaces of the protrusions having the gold film. Therefore, the enzyme-free glucose detection chip disclosed in the present invention can be obtained.

When the foregoing preparation method is applied to a base material having a large size, a plurality of enzyme-free glucose detection chips can be fabricated at the same time on the base material. That is, the base material may be cut into a plurality of substrates having suitable sizes, and each substrate has one detection portion.

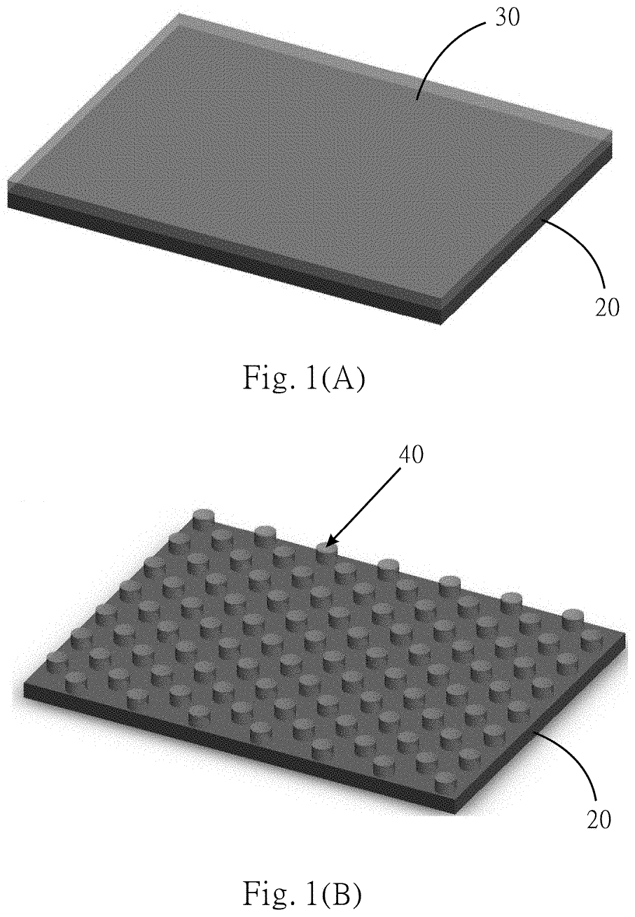

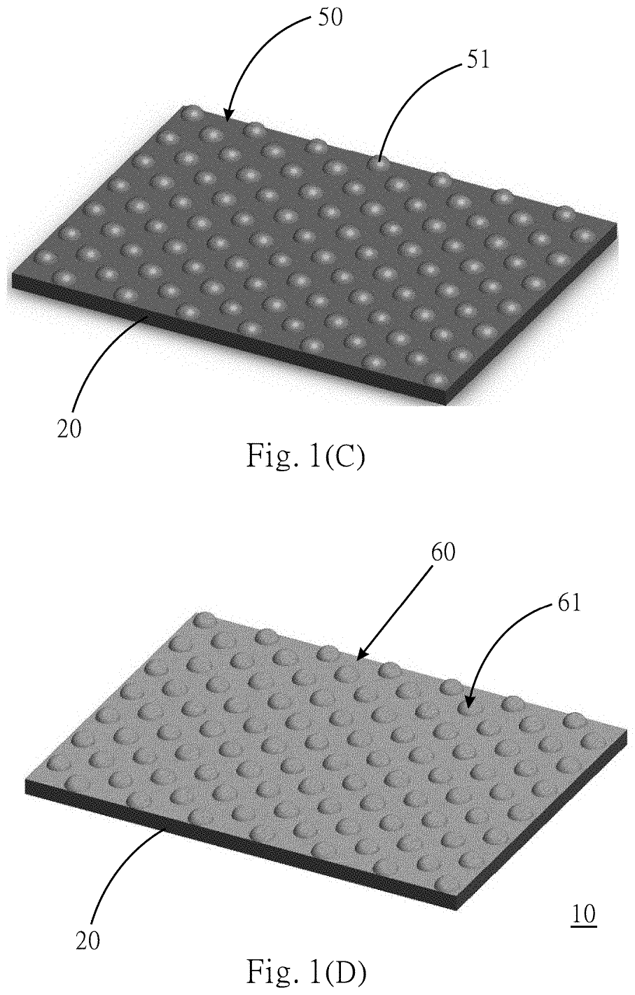

Referring to FIG. 1, disclosed in an embodiment of the present invention is continuous manufacturing of an enzyme-free glucose detection chip (10) disclosed in the present invention. The included steps are as follows.

(I) Cleaning a Silicon Chip and a Photoresist Coating

A substrate (20) formed of a silicon chip having a predetermined size is taken, sequentially cleaned in acetone, alcohol, and deionized water with an ultrasound wave, and blown with nitrogen gas, and residual moisture is removed with a heating plate.

First, the substrate (20) is coated with hexamethyldisilane (HMDS), so as to increase the adhesion between a surface of the substrate and a photoresist coating. Next, the surface of the substrate (20) is coated with a photoresist through spin coating, forming a photoresist layer (30).

In an embodiment of the present invention, the photoresist is an AZ1518 positive photoresist. Spin coating parameters used for the AZ1518 positive photoresist are as follows: a spinning speed at a first level is 500 rpm, and a spinning time is 10 seconds; a spinning speed at a second level is 1500 rpm, and a spinning time is 40 seconds; and a coating thickness of the photoresist layer is approximately 1 μm to 10 μm, and is preferably 3 μm.

Finally, the substrate having the photoresist layer is dried in a manner such as baking.

(II) Exposure and Development

A mask aligner is used to transfer a required pattern to the photoresist layer (30) on the substrate (20). Subsequently, a 2.38% THAM development solution is used for treatment. As a result, a silicon chip having a columnar photoresist array (40) is obtained, as shown in FIG. 1B.

In an embodiment of the present invention, the model of the mask aligner is EVG620, the intensity of a light source is approximately 22 mW/cm² (i-line), an exposure time is approximately 7.5 seconds, and a development time is approximately 50 seconds.

A development condition is confirmed with an optical microscope.

(III) Thermal Melting Step

By means of gradually increasing environmental temperature to be higher than a glass-transition temperature of the photoresist, and based on the influence of surface tension, the columnar photoresist array (40) gradually becomes a semispherical photoresist array (50) in a thermal melting process, the semispherical photoresist array (50) having a plurality of protrusions (51), as shown in FIG. 1C.

In an embodiment of the present invention, the glass-transition temperature of the AZ1518 positive photoresist is 130° C., and the environmental temperature is gradually increased to 150° C. within 5 minutes.

Moreover, in a process of fabricating the enzyme-free glucose detection chip disclosed in the present invention, the thermal melting step is to change a columnar photoresist array into a semispherical photoresist array. However, the thermal melting step is not an essential step in fabrication of the enzyme-free glucose chip, that is, if a photoresist array to be used is not semispherical, this step may be omitted.

(IV) Sputtering of a Gold Film and Deposition of Gold Nanoparticles

After the thermal melting step, a film (60) is sputtered on a surface of the substrate (20) having the semispherical photoresist array (50) through a direct-current sputtering method, and then the gold nanoparticles (61) are evenly disposed on surfaces of the semispherical photoresist arrays, so as to obtain the enzyme-free glucose detection chip disclosed in the present invention, as shown in FIG. 1D. The film (60) can be made by gold or a mixture of gold and platinum, for example, when the film is made by the mixture of gold and platinum, the ratio (mol/v) of platinum to gold is 1:2 to 4:1.

In an embodiment of the present invention, a gold film layer is sputtered on the substrate with a direct-current sputtering machine. Sputtering conditions are as follows: A pressure is 0.08 millibar, a current is 30 milliampere, and a treatment time is 135 seconds. To ensure the evenness of the sputtered gold film, a sample is usually heated to 120° C. at a heating rate of 5° C./minute, the temperature is kept for approximately 80 minutes, and eventually the sample is cooled to room temperature.

Furthermore, to ensure the consistency of a sensing region, a packaging step also needs to be added. The packaging step can be performed before or after gold nanoparticles are deposited.

In an embodiment of the present invention, packaging is performed by using a screen printing technology. In particular, a base material is taken; the base material is treated according to the photolithography, the photoresist thermal melting method, and the step of sputtering of the film, and the like in the foregoing embodiment; the base material is then cut into a plurality of substrates, where each substrate has a detection portion. The cut base material is fixed by an adhesive tape. Subsequently, a screen plate having a certain pattern is aligned with the base material. After ink is applied so that the ink covers a region other than a sensing portion, the screen plate is removed. After the ink is dried, the cut substrates are then removed one by one from the adhesive tape. Then, a subsequent step such as deposition of gold nanoparticles is performed.

In an embodiment of the present invention, a conductive silver wire is first disposed on a glass slide and used as a lead. A sealing film having a predetermined size and having a hole is then bonded to the enzyme-free glucose detection chip, where the hole on the sealing film corresponds to the detection portion, and the sealing film is used to cover a block of a non-detection portion on the enzyme-free glucose detection chip and the glass slide.

In an embodiment of the present invention, after sputtering of the gold film layer is completed, the surfaces of the protrusions are modified with a molecular layer. For example, the surfaces of the protrusions are modified with an APTMS molecule solution.

The gold nanoparticles disclosed in the present invention are prepared by using a technology belonging to the technical field of the present invention and well known by a person of ordinary skill. For detailed technical content, reference may be made to I.-C. Ni, S.-C. Yang, C.-W. Jiang, C.-S. Luo, W. Kuo, K.-J. Lin, et al., Formation mechanism, patterning, and physical properties of gold-nanoparticle films assembled by an interaction-controlled centrifugal method, The Journal of Physical Chemistry C, 116(2012) 8095-101. Details are not described herein.

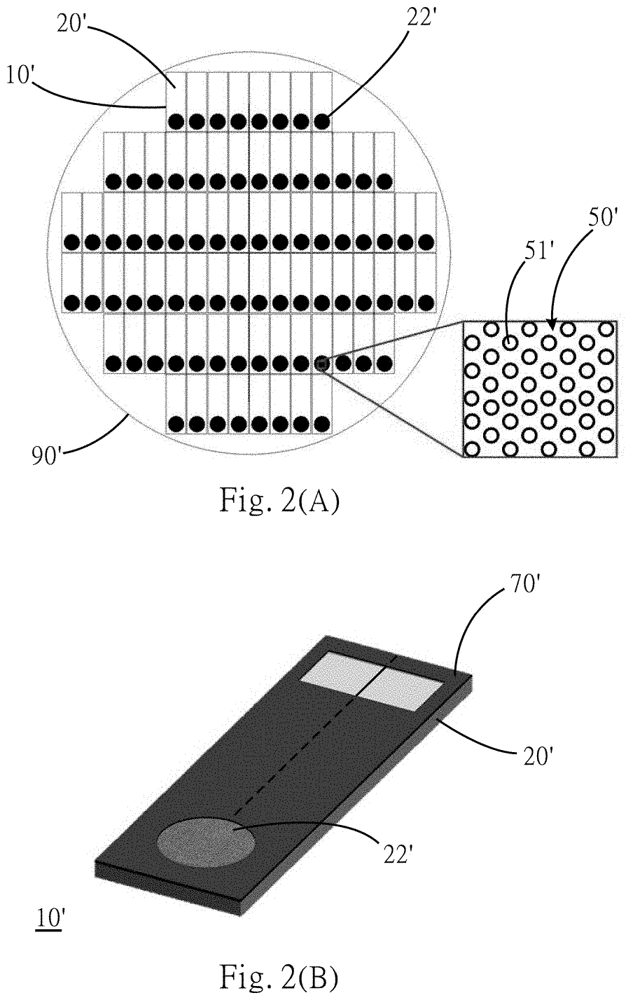

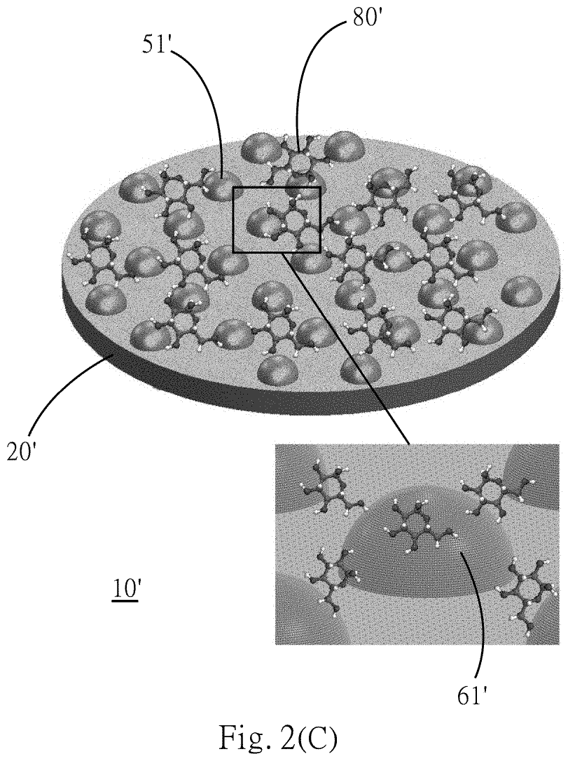

Referring to FIG. 2, FIG. 2 shows a preparation procedure of another preferred embodiment disclosed in the present invention. The base material (90′) is an 8-inch silicon chip (the thickness is approximately 700 μm; Phoenix Silicon International, Taiwan). Approximately 80 circular detection portions (22′) are disposed on the base material (90′), where the diameter of a detection portion is approximately 8 millimeters. A columnar-photoresist array is etched on each detection portion by using a photolithography technology, the columnar-photoresist array having over 2 millions of columnar photoresists densely arranged in a hexagonal shape. Subsequently, the columnar photoresist array becomes a semispherical photoresist array (50′) having a plurality of protrusions (51′) by using a thermal melting technology, where the diameter of each protrusion (51′) and a gap between the protrusions (51′) are both 3 μm. A surface of the base material (90′) having the detection portion (22′) is first sputtered with a gold film and modified, and the base material (90′) is then cut into a plurality of substrates (20′), where each substrate (20′) has a detection portion (22′). The cut base material (90′) is packaged by using a screen printing technology, so that a packaging layer (70′) covers a region other than the detection portion (22′) on each substrate (20′). After packaging is completed, gold nanoparticles (61′) are deposited on the protrusions (51′), to obtain a plurality of enzyme-free glucose detection chips disclosed in the present invention (10′). Further, referring to FIG. 2C, for the enzyme-free glucose detection chip (10′), through reactions between the gold nanoparticles (61′) on the protrusions (51′) and glucose (80′), an effect of detecting glucose in an enzyme-free manner can be achieved.

In an embodiment of the present invention, if the protrusions are not arranged in a hexagonal shape, efficacy of the present invention can also be achieved.

In addition, in an embodiment disclosed in the present invention, a size of the base material and a size of the detection portion can be changed according to a manufacturing requirement. For example, a 6-inch silicon chip may be used as the base material, and preferably 40 detection portions are disposed on the 6-inch chip.

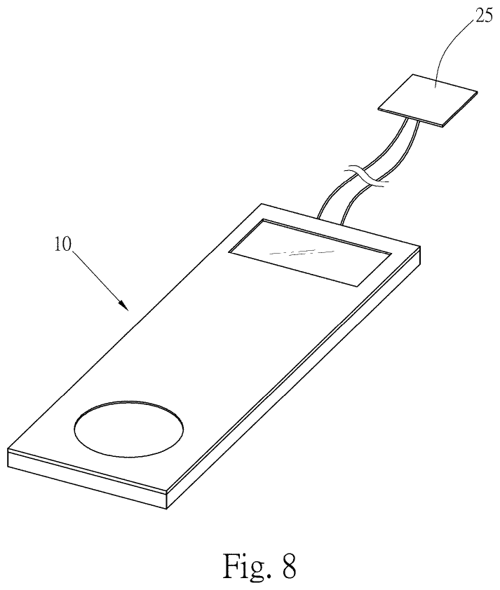

In another embodiment of this invention, the enzyme-free glucose detection chip further includes a power generation portion (25) made by alloy of gold and platinum that can use the glucose of the blood tissue as fuel to generate the electricity by oxidation reaction, so that the enzyme-free glucose detection chip can be implanted into the blood tissue of a subject without the exterior power. For example, the structure of the enzyme-free glucose detection chip is shown as FIG. 8.

The structure and performance of the enzyme-free glucose detection chip disclosed in the present invention are described below through several examples with reference to the drawings. In an example below, where an SP-150 potentiostat (Bio-Logic, USA) is used as an electrochemical detection instrument.

### Example 1: Observation of a Process of Fabricating an Enzyme-Free Glucose Detection Chip

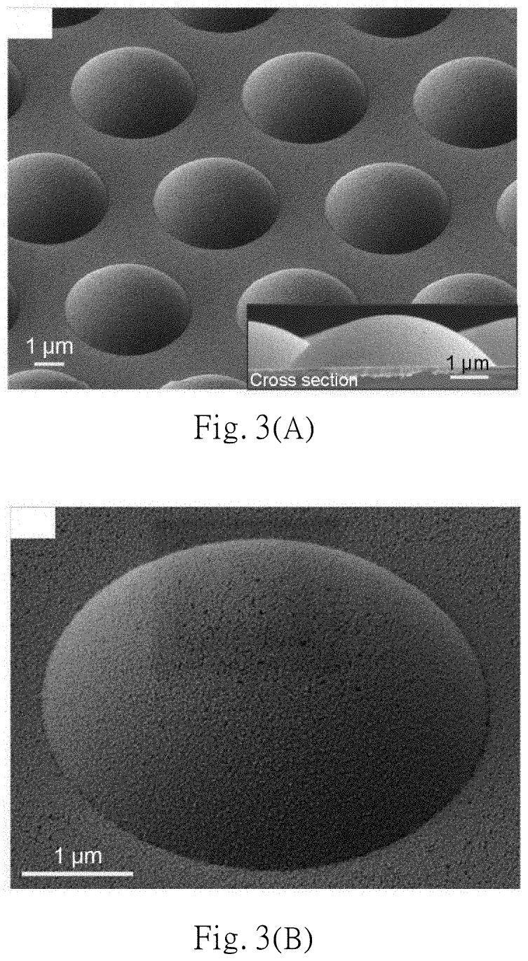

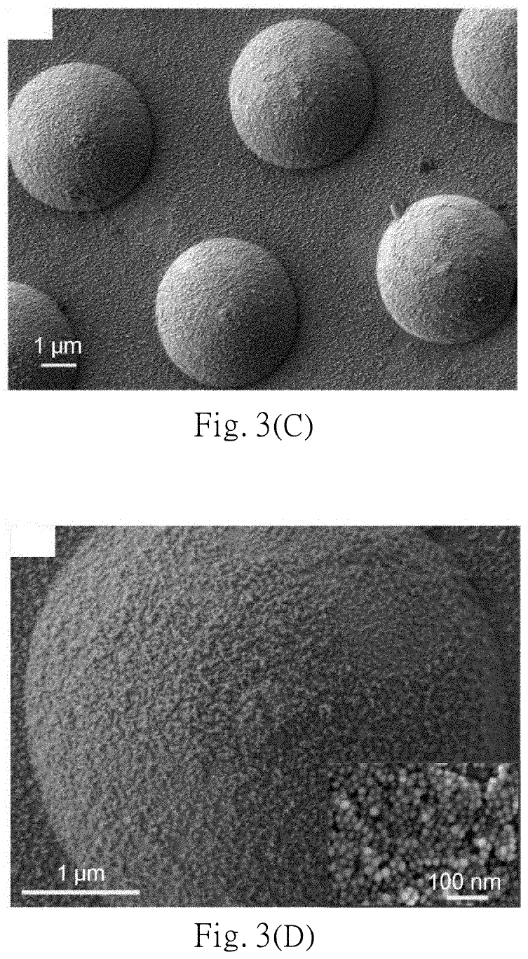

Referring to FIG. 3, the appearance of protrusions of the semispherical array of the enzyme-free glucose detection chip disclosed in the present invention is observed by using a field emission electron microscope (field emission gun scanning electron microscopy; JSM-6700F, JEOL, Japan).

FIG. 3A shows that after thermal melting succeeds, a columnar protrusion array formed by using a photolithography technology is converted into a semispherical protrusion array. The height of each semispherical protrusion is approximately 2 micrometers, as shown in a square box in FIG. 3A, and the semispherical protrusions are evenly arranged.

FIG. 3B shows that the diameter of each protrusion is approximately 4 micrometers. Because of surface tension and hydrophobicity, after the thermal melting step, a patterned region of the positive photoresist layer is slightly expanded.

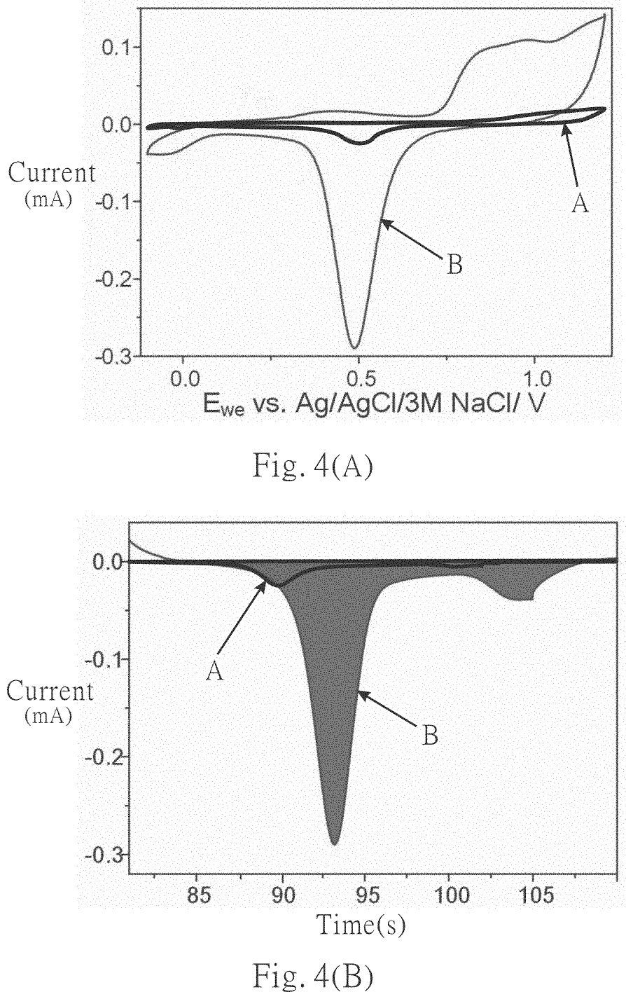

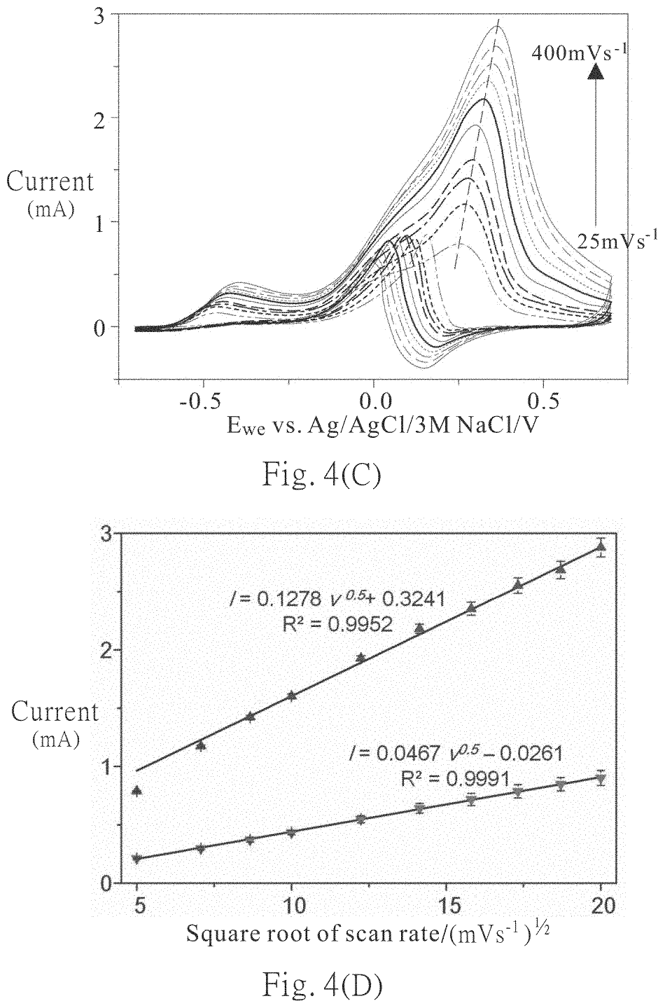

Referring to FIG. 3C and FIG. 3D, FIG. 3C and FIG. 3D show that gold nanoparticles are evenly deposited on the surfaces of the protrusions, and the shape of each semispherical protrusion is still maintained complete. The size of a gold nanoparticle is approximately 20 nm (as shown in a square box in FIG. 4D).

### Example 2: Analyzation of the Enzyme-Free Glucose Detection Chip Disclosed in the Present Invention Through Cyclic Voltammetry

In this example, an actual sensing region of the protrusions of the enzyme-free glucose detection chip disclosed in the present invention is estimated through the cyclic voltammetry in a 0.1M phosphate buffer solution (the ph value is 7.0) at a scan rate of 50 mV·s−1. The result is shown in FIG. 4A and FIG. 4B.

Referring to FIG. 4B, a current for a region under a horizontal line is 0, representing a total charge required for complete reduction of an electrode. Therefore, FIG. 4B shows that, for the protrusions disclosed in the present invention, the total charge corresponding to the area of the region under the horizontal line is 1120.6 μC. On the basis that a gold electrode of 1 square centimeter requires a total charge of 390 μC to form gold oxide, it is estimated that an effective sensing region of protrusions disclosed in the present invention is a cyclic voltammogram of a normal gold electrode of 2.873 square centimeters (1120.6 μC/390 μC). A geometrical region under the horizontal line for the protrusions disclosed in the present invention is approximately 10.2 times as large as that for a normal gold electrode.

Moreover, at different scan rates: 25, 50, 75, 100, 150, 200, 250, 300, 350, and 400 mV·s−1, in a 0.1 M sodium hydroxide solution containing 5.56 mM of glucose electrolytes, the protrusions disclosed in the present invention are observed through cyclic voltammetry. The result is shown in FIG. 4C. As can be seen from the result in FIG. 4C, as the scan rate increases, a peak current and a peak potential also increase. A typical diffusion reaction may be confirmed through the following Randles-Sevcik formula:

iₚ=2.69×10⁵×n^{3/2}×A×C×D^{1/2}×V^{1/2}

where iₚ represents the value of a peak current (A); n represents the number of electrons appearing in a half reaction for redox electron pairs; A is the area (square centimeter) of an electrode; C is the concentration (mol/cm³) of an analyte; and D is a diffusion rate (V/s) of the analyte. Suppose A, C, and D are all fixed, iₚ is directly proportional to a square root of the scan rate.

A linear relationship between the peak current and the scan rate of protrusions disclosed in the present invention is shown in FIG. 4D. As can be seen from the result in FIG. 4D, the peak current and the square root of the scan rate are highly and linearly correlated. Therefore, the enzyme-free glucose detection chip disclosed in the present invention manifests a typical diffusion-controlled electrochemical behavior, and may be suitable for an application including actual quantitative analysis. Moreover, a slope of a Randles-Sevcik line for the enzyme-free glucose detection chip disclosed in the present invention is approximately 2.7 times as large as that for the normal gold electrode, that is, the enzyme-free glucose detection chip disclosed in the present invention has better mass transfer efficiency.

### Example 3: Analyzation of the Sensitivity of the Enzyme-Free Glucose Detection Chip Disclosed in the Present Invention

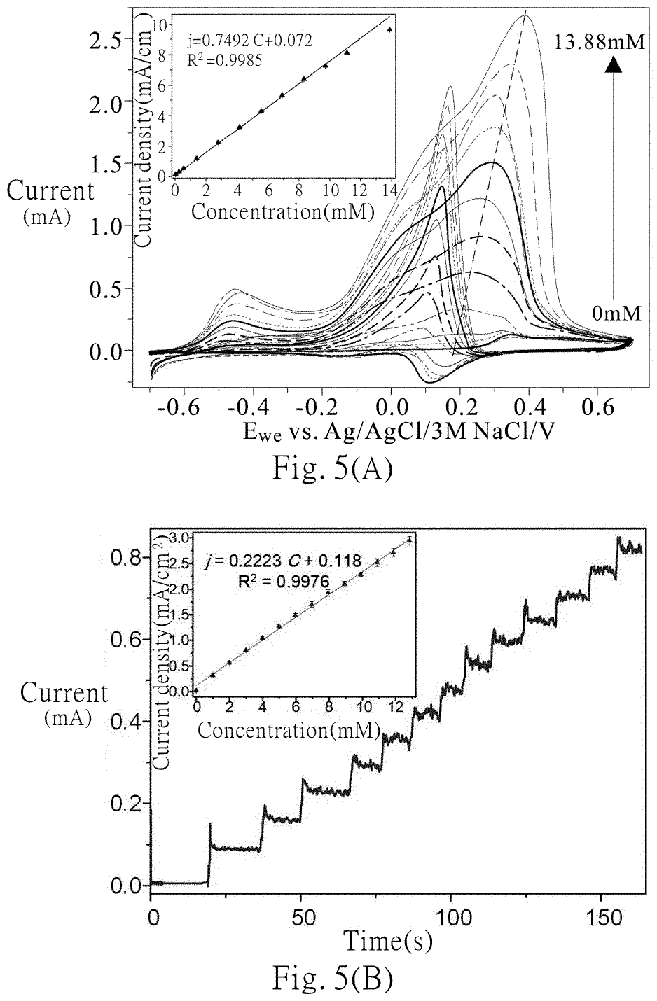

Through cyclic voltammetry, at a scan rate of 50 mV·s−1, on condition that a 0.1 M sodium hydroxide solution contains different concentration of glucose: 0, 0.06, 0.28, 0.56, 1.39, 2.78, 4.16, 5.56, 6.94, 8.32, 9.71, 11.10, and 13.89 mM, a cyclic voltammogram shown in FIG. 5A is obtained, where each measurement is repeated 5 times.

Moreover, 1 mM glucose is continuously added to the enzyme-free glucose detection chip disclosed in the present invention, and a current analysis method is used to perform analysis. The result is shown in FIG. 5B. In particular, the enzyme-free glucose detection chip disclosed in the present invention is immersed in a 0.1 M sodium hydroxide solution that is being continuously stirred, and a constant potential of 0.1 V is provided. Subsequently, 1 mM of glucose is regularly added to the sodium hydroxide solution, and a trajectory is recorded with a treatment time. An ampere current increases rapidly as glucose is added each time. In addition, in this analysis, currents measured in the steps and glucose concentration related to the currents are rearranged. Thus, a standard curve shown in a square box in FIG. 5B is depicted.

As can be seen from the result in FIG. 5A, the range of linear detection is 55.56 μM to 13.89 mM, and a high R² value is 0.9985. It can be calculated according to the result that the sensitivity of the enzyme-free glucose detection chip disclosed in the present invention is 749.2 μA·mM−1·cm−2, and a limit of detection is 9 μM. As can be seen from the result in FIG. 5B, the standard curve is linearly and directly proportional to the glucose concentration range within 1 mM to 13 mM, having a correlation coefficient of 0.9976, and the sensitivity is 222.3 μA·mM−1·cm−2.

As can be seen according to the foregoing result, the high sensitivity of the enzyme-free glucose detection chip disclosed in the present invention is due to a relatively large effective sensing area of the enzyme-free glucose detection chip, which can oxidize a significant amount of glucose.

### Example 4: Analyzation of the Selectivity of the Enzyme-Free Glucose Detection Chip Disclosed in the Present Invention

Other substances such as ascorbic acid in human blood may interfere with the performance of a glucose detection instrument. The reason is that in a normal human body, the glucose concentration (3 mM to 8 mM) is much higher than the concentration (˜0.1 mM) of the interfering substance. Therefore, the selectivity of the enzyme-free glucose detection chip disclosed in the present invention can be detected by reducing the concentration ratio of the glucose to the ascorbic acid to 10.

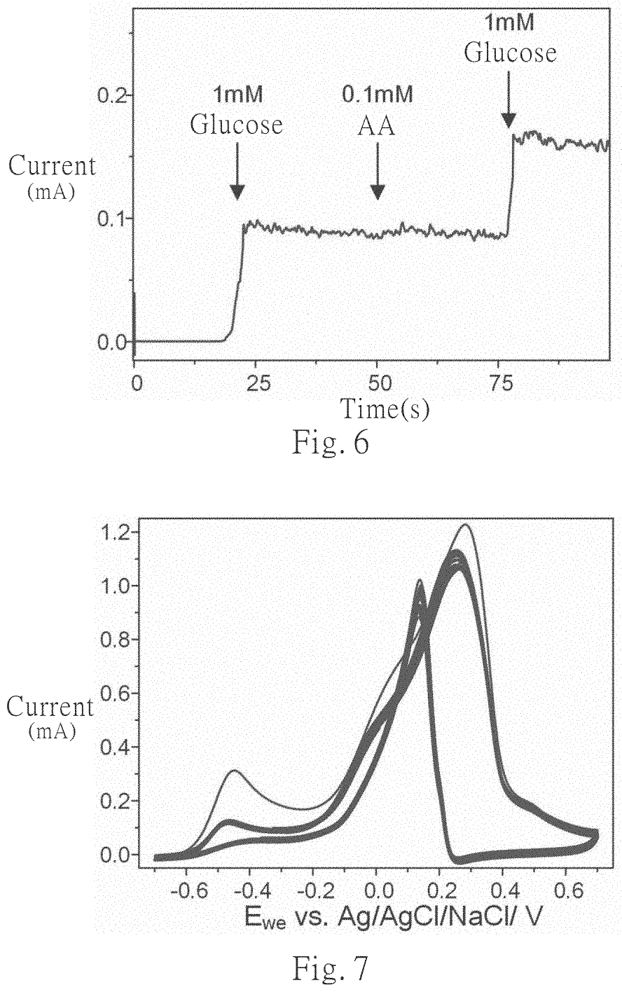

1 mM of glucose, 0.1 mM of ascorbic acid, and a 0.1 M of sodium hydroxide solution containing 1 mM of glucose are sequentially injected onto the enzyme-free glucose detection chip disclosed in the present invention in a condition of an operable potential of 0.1 V, to perform detection. The result is shown in FIG. 6. The result in FIG. 6 shows that the enzyme-free glucose detection chip disclosed in the present invention is hardly interfered with by the ascorbic acid.

### Example 5: Analyzation of the Stability of the Enzyme-Free Glucose Detection Chip Disclosed in the Present Invention

A 0.1 M sodium hydroxide solution containing 5.56 mM of glucose is used as an electrolyte. The stability of the enzyme-free glucose detection chip disclosed in the present invention is detected cyclically through 20 times of cyclic voltammetry. The result is shown in FIG. 7.

Because the electrolyte is not stirred, the oxidization reaction of glucose is the most intense and the most rapid during the first time of scan. Therefore, a wave peak obtained at the first time of cyclic voltammetry scan is higher than wave peaks obtained in subsequent scans. After the first time of scan, the glucose near the surface of the electrode (that is, the protrusion) is reacted and consumed, so that reactions observed in subsequent scans are reduced. However, after the second time of scan, a wave peak current changes very slightly, because the glucose is continuously diffused to the surface of the electrode, and a diffusion speed and a reaction speed are nearly the same, so that reactions change slightly in subsequent scans. The result shows that, the enzyme-free glucose detection chip disclosed in the present invention has high stability.

In addition, after the enzyme-free glucose detection chip disclosed in the present invention is stored in air at room temperature for two months, the detection performance of the enzyme-free glucose detection chip still remains unchanged. In other words, the enzyme-free glucose detection chip disclosed in the present invention can be stored easily, and is not deteriorated or modified under the influence of an external environmental factor, thereby completely overcoming the disadvantage that a conventional glucose detection test strip is deteriorated because of an environmental factor.

### Example 6: Efficacy Comparison Result

Other enzyme-free glucose sensors found in the documents are shown in Table 1 below:

TABLE 1
Different enzyme-free glucose sensors

| No. | Data source |
|---|---|
| 1 | D. Feng, F. Wang, Z. Chen, Electrochemical glucose sensor |
| | based on one-step construction of gold nanoparticle-chitosan |
| | composite film, Sensors and Actuators B: Chemical, 138(2009) |
| | 539-44. |
| 2 | J. Li, Z. Wang, P. Li, N. Zong, F. Li, A sensitive non-enzyme |
| | sensing platform for glucose based on boronic acid-diol |
| | binding, Sensors and Actuators B: Chemical, 161(2012) 832-7. |
| 3 | F. Xu, K. Cui, Y. Sun, C. Guo, Z. Liu, Y. Zhang, et al., |
| | Facile synthesis of urchin-like gold submicrostructures for |
| | nonenzymatic glucose sensing, Talanta, 82(2010) 1845-52. |
| 4 | L. Chen, X. Lang, T. Fujita, M. Chen, Nanoporous gold for |
| | enzyme-free electrochemical glucose sensors, Scripta |
| | Materialia, 65(2011) 17-20. |
| 5 | H. Shu, L. Cao, G. Chang, H. He, Y. Zhang, Y. He, Direct |
| | electrodeposition of gold nanostructures onto glassy carbon |
| | electrodes for non-enzymatic detection of glucose, |
| | Electrochimica Acta, 132(2014) 524-32. |
| 6 | S. Cherevko, C.-H. Chung, Gold nanowire array electrode for |
| | non-enzymatic voltammetric and amperometric glucose |
| | detection, Sensors and Actuators B: Chemical, 142(2009) |
| | 216-23. |
| 7 | C. Shen, J. Su, X. Li, J. Luo, M. Yang, Electrochemical |
| | sensing platform based on Pd—Au bimetallic cluster for |
| | non-enzymatic detection of glucose, Sensors and Actuators B: |
| | Chemical, 209(2015) 695-700. |

The sensitivity, limits of detection (LOD), and linear ranges of the foregoing enzyme-free glucose detectors numbered 1 to 7 and the enzyme-free glucose detection chip disclosed in the present invention are compared, and the result is shown in Table 2 below.

TABLE 2
Comparison of sensitivity, limits of detection, and linear
ranges of the enzyme-free glucose detection tools

| | | | Limit of | |
|---|---|---|---|---|
| | | Sensitivity | detection | Linear range |
| No. | Electrode | (μA · mM − 1 · cm − 2) | (μM) | (mM) |
| 1 | Chitosan/gold | Not available | 370 | 0.4-10.7 |
| | nanoparticles/glassy carbon | | | |
| | electrode (Chitosan/GNPs/GCE) | | | |
| 2 | Gold | Not available | 0.05 | 0.0001-0.0135 |
| | nanoparticles-platinum/glass | | | |
| | electrode GNPs-PB/GE) | | | |
| 3 | Nafion-UGS/glassy carbon | 16.8 | 10 | 0.2-13.2 |
| | electrode Nafion-Urchin-like gold | | | |
| | submicrostructures/GCE) | | | |
| 4 | Nanoporous gold (Nanoporous | 20.1 | 3 | 10.-18.0 |
| | Au) | | | |
| 5 | Gold nanostructure/glassy | 19.07 | 0.05 | 0.1-25 |
| | carbon electrode (Gold | | | |
| | nanostructure/GCE) | | | |
| 6 | Gold nanowire array (Gold | 309 | 50 | 0.5-14 |
| | nanowire array) | | | |
| 7 | Palladium-gold cluster (Pd—Au | 75.3 | 50 | 0.1-30 |
| | cluster) | | | |
| Present | Sensor having a micro/nano | 749.2 | 9 | 0.0556-13.89 |
| invention | composite structure | | | |

As can be seen from the result in Table 2, the stability, sensitivity, limits of detection, and linear ranges of the enzyme-free glucose detection chip disclosed in the present invention are all significantly superior to those of current existing enzyme-free glucose detection tools.

### Example 7: Detecting the Glucose in a Neutral Environment

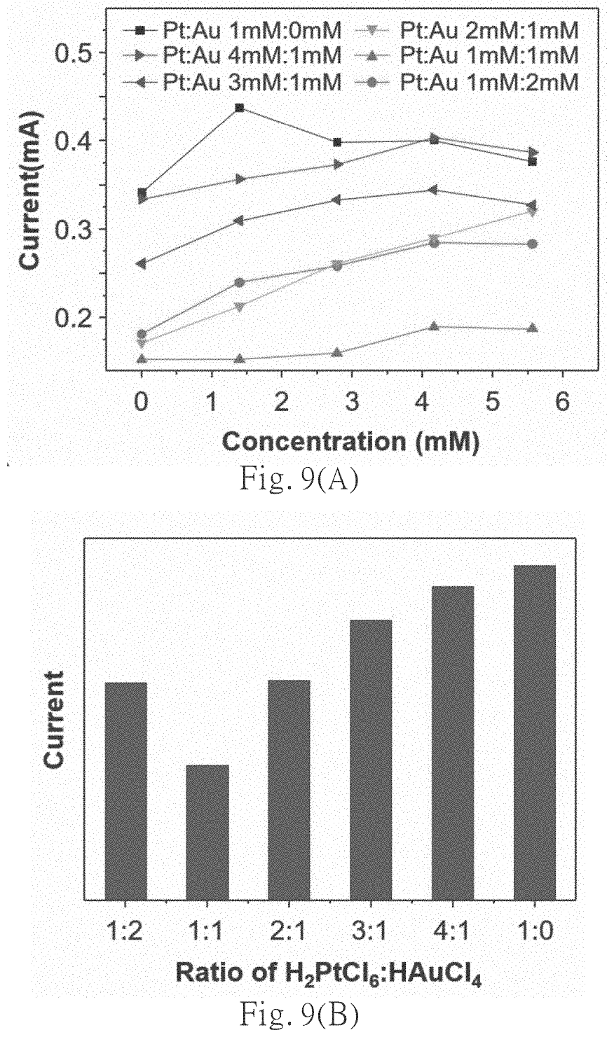

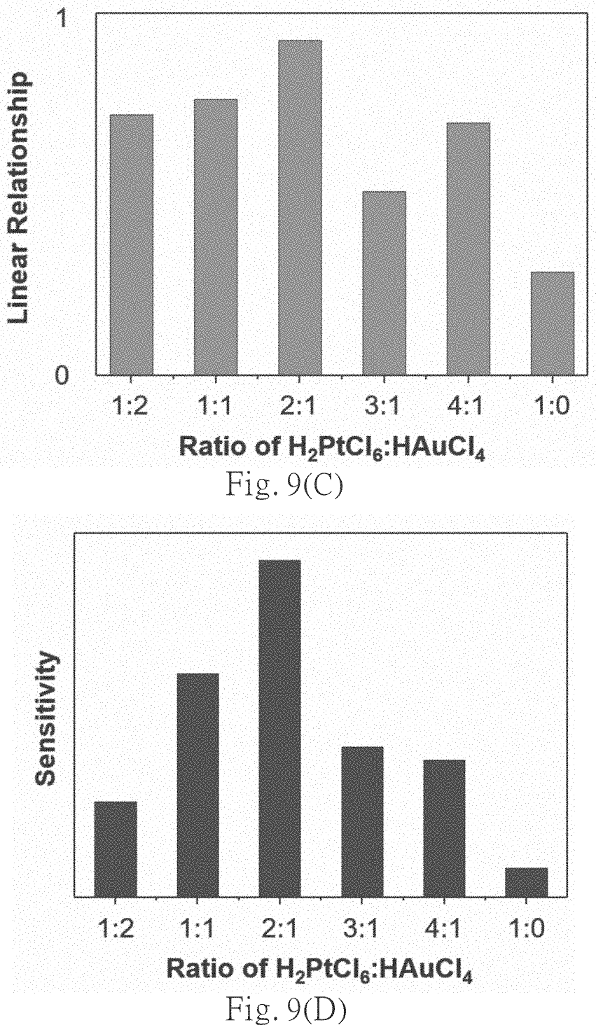

Following the content of the examples 1 to 7, it provided the enzyme-free glucose detection chips of this invention to detect the glucose in the neutral environment, wherein the conductive layer of each enzyme-free glucose detection chip was made in different mixing ratio (mol/v) of platinum to gold: 1:0, 4:1, 3:1, 2:1, 1:1, 1:2. The results were shown in FIGS. 9A to 9D.

According to the FIGS. 9A to 9D, the enzyme-free glucose detection chip with the conductive layer made by mixing of platinum and gold can be detect the glucose in the neutral environment, especially, when the mixing ratio (mol/v) of platinum to gold is 2:1, the enzyme-free glucose detection chip has better stability and sensitivity for detecting glucose. Therefore, when the conductive layer made by platinum and gold, the enzyme-free glucose detection chip of the invention can be used in the neutral environment to achieve the goal of detecting the blood sugar of the blood. In other words, it does not need to pretreat the blood's PH value to become alkaline or adjust the testing environment to become alkaline.

As can be seen from the foregoing description, the enzyme-free glucose detection chip disclosed in the present invention has advantages of a simple fabrication process, a low cost, and easy storage.

The above examples are merely provided to describe the present invention, and any simple modification or change made to the embodiments in the specification by a person skilled in the art without departing from the spirit of the present invention should be covered by the patent scope of the claims of the present application.

What is claimed is:

1. An enzyme-free glucose detection chip for detecting blood sugar level of a blood directly in a neutral environment, the enzyme-free glucose detection chip comprising:
a substrate;
a detection portion, disposed on an end surface of the substrate;
a plurality of protrusions, disposed at the detection portion;
a conductive layer, made by mixing gold and platinum in a predeterminate ratio and disposed on a surface of the substrate having the protrusions; and
a plurality of gold nanoparticles, dispersed on surfaces of the protrusions.

2. The enzyme-free glucose detection chip of claim 1, wherein the predeterminate ratio (mol/v) of platinum to gold is 1:2 to 4:1.

3. The enzyme-free glucose detection chip of claim 2, wherein the predeterminate ratio (mol/v) of platinum to gold is 2:1.

4. The enzyme-free glucose detection chip of claim 1, wherein the blood is from a subject.

5. The enzyme-free glucose detection chip of claim 1, wherein the blood is from a subject.

6. The enzyme-free glucose detection chip of claim 1, wherein the enzyme-free glucose detection chip further including a power generation portion made by alloy of gold and platinum.

7. The enzyme-free glucose detection chip of claim 1, the enzyme-free glucose detection chip is implanted into a body.

8. The enzyme-free glucose detection chip of claim 1, each protrusion is semispherical.

9. The enzyme-free glucose detection chip of claim 1, wherein each protrusion is columnar.

10. The enzyme-free glucose detection chip of claim 1, wherein each protrusion has a micrometer-level size.

11. The enzyme-free c glucose detection hip of claim 2, wherein each protrusion has a diameter between 1 micrometer and 20 micrometers.

12. The enzyme-free glucose detection chip of claim 1, wherein each gold nanoparticle has a diameter between 2 nanometers and 100 nanometers.
